(19) United States
(12) Patent Application Publication
(10) Pub. No.: US 2022/0178950 A1
(43) Pub. Date: Jun. 9, 2022

(54) DISCOVERY AND VALIDATION OF AN EARLY POST-TRANSPLANT BIOMARKER PREDICTIVE OF CHRONIC KIDNEY DISEASE IN LIVER TRANSPLANT RECIPIENTS

(71) Applicant: Northwestern University, Evanston, IL (US)

(72) Inventors: Josh Levitsky, Evanston, IL (US); Michael M. Abecassis, Highland Park, IL (US)

(21) Appl. No.: 17/681,315

(22) Filed: Feb. 25, 2022

Related U.S. Application Data
(63) Continuation of application No. 16/600,135, filed on Oct. 11, 2019, now abandoned.
(60) Provisional application No. 62/744,444, filed on Oct. 11, 2018.

(51) Int. Cl. G01N 33/70; G01N 33/68; G16H 50/30

(52) U.S. Cl. CPC G01N 33/70; G01N 33/6803; G16H 50/30; G01N 2800/347; G01N 2800/245; G01N 2800/52; G01N 2800/56; G01N 2800/60

(57) ABSTRACT

Disclosed are methods for predicting, inhibiting, and treating chronic kidney disease (CKD) in a subject that that is preparing to undergo a liver transplant. The methods may include detecting a protein biomarker in a serum sample from the subject prior to the subject being administered a liver transplant procedure and/or after the subject has been administered the liver transplant procedure. Suitable protein markers for the disclosed methods may include but are not limited to osteopontin (OPN) and/or tissue inhibitor of metalloproteases 1 (TIMP1).

### CROSS-REFERENCE TO RELATED PATENT APPLICATIONS

The present application is a continuation of U.S. application Ser. No. 16/600,135, filed Oct. 11, 2019, which claims the benefit of priority under 35 U.S.C. § 119(e) to U.S. Provisional Application No. 62/744,444, filed on Oct. 11, 2018, the content of each is incorporated herein by reference in their entireties.

### STATEMENT REGARDING FEDERALLY SPONSORED RESEARCH OR DEVELOPMENT

This invention was made with government support under AI084146 and AI113916 awarded by the National Institutes of Health. The government has certain rights in the invention.

### BACKGROUND

The field of the invention relates to methods for predicting, inhibiting, and treating chronic kidney disease in subjects that that are preparing to undergo a liver transplant.

A significant proportion of patients develop chronic kidney disease (CKD) after liver transplantation (LT). The only current strategy is to employ renal protective strategies when kidney function obviously deteriorates. There is a need for a technology that predicts the likelihood or onset of CKD early before kidney deterioration to prevent renal dysfunction altogether instead of waiting for CKD to occur. We have developed clinical/protein models to predict glomerular filtration rate (GFR) deterioration in recipients with normal GFR early after LT.

Using independent cohorts, we analyzed protocol serum/plasma samples from liver transplant recipients with preserved GFR (>60 ml/min/1.73 m²) at month 3 and samples from liver transplant recipient who had GFR deterioration versus preservation at 1 year or year 5 GFR. We also studied serial renal injury protein levels and GFR during the first year.

Using one cohort (n=61), a year 1 GFR model was constructed from 7 clinical and 16 renal injury protein candidate variables. Hepatitis C virus (HCV) infection and protein levels of 02-microglobulin and CD40 predicted early GFR deterioration, defined as >10% persistent decline over year 1 (AUC 0.814). We observed good validation of this model (AUC 0.801) in a second sub-cohort (n=50) that met this same GFR definition of deterioration. Several proteins, including β2-microglobulin, α1-microglobulin, cystatin C, trefoil factor 3, thrombomodulin, and uromodulin, correlated with deterioration in GFR over the first year post-LT in a linear mixed model.

A year 5 GFR model that predicts longer term, more advanced chronic kidney disease (CKD) (<45 ml/min/1.73 m²) was constructed based on month 3 samples (n=105). This model, which includes age, male sex, cystatin-C and MCSF-1 (AUC 0.866), confirms that early renal injury protein levels can also predict late GFR deterioration.

We have developed and validated a clinical/protein model at 3 months post-LT (in preserved GFR recipients) that can predict renal deterioration at 1 year with reasonable accuracy. In addition, we report on a 5 year model and serial increases in several kidney injury proteins with GFR deterioration in the first year. These approaches may identify appropriate candidates for proactive renal sparing strategies early post LT.

The technology can be used to identify those at highest risk for kidney problems after a liver transplant before they occur and design strategies, such as immunosuppression medication selection and renal protective medications, in order to protect kidney function in those patients. The disclosed technology is advantageous because it clinicians to predict the onset of CKD before kidney deterioration to prevent renal dysfunction altogether instead of waiting for kidney deterioration to occur.

### SUMMARY

Disclosed are methods for predicting, inhibiting, and treating chronic kidney disease (CKD) in a subject that that is preparing to undergo a liver transplant. The methods may include detecting a protein biomarker in a serum sample from the subject prior to the subject being administered a liver transplant procedure and/or after the subject has been administered the liver transplant procedure. Suitable protein markers for the disclosed methods may include but are not limited to osteopontin (OPN) and/or tissue inhibitor of metalloproteases 1 (TIMP1).

Based on the detected levels of the protein biomarker in the serum sample of the subject, the subject optionally may be administered additional treatment. In some embodiments, the subject may be administered a simultaneous liver transplant and kidney transplant. In other embodiments, the subject may be administered therapy in order to treat or inhibit chronic kidney disease (CKD) or to reduce the rate of progression of CKD, before and/or after the subject being administered the liver transplant procedure. In further embodiments, the subject may be administered therapy in order to reduce the nephrotoxic effects of immunosuppressive therapy that is administered before, during, or after the liver transplant procedure.

### BRIEF DESCRIPTION OF THE FIGURES

FIG. 1A: Absolute Levels Of Osteopontin Across AKI Groups, Per Estimated GFR. (1) eGFR<30 ml/min/1.73 m² pre-LT and eGFR<30 ml/min/1.73 m² 4-12 weeks after LT (irreversible AKI=iAKI), (2) eGFR<30 ml/min/1.73 m² pre-LT and >50 ml/min/1.73 m² 4-12 weeks after LT (reversible AKI=rAKI), (3) eGFR>50 ml/min/1.73 m² prior to LT and >50 ml/min/1.73 m² 4-12 weeks after LT (normal=nAKI). (4) eGFR<30 ml/min/1.73 m² pre-LT and eGFR 30-50 ml/min/1.73 m² after LT (partial=pAKI).

FIG. 1B: Absolute Levels Of TIMP1 Across AKI Groups, Per Estimated GFR.
(1) eGFR<30 ml/min/1.73 m² pre-LT and eGFR<30 ml/min/1.73 m² 4-12 weeks after LT (irreversible AKI=iAKI), (2) eGFR<30 ml/min/1.73 m² pre-LT and >50 ml/min/1.73 m² 4-12 weeks after LT (reversible AKI=rAKI), (3) eGFR>50 ml/min/1.73 m² prior to LT and >50 ml/min/1.73 m² 4-12 weeks after LT (normal=nAKI). (4) eGFR<30 ml/min/1.73 m² pre-LT and eGFR 30-50 ml/min/1.73 m² after LT (partial=pAKI).

FIG. 2A: Change In OPN Levels Before and After Liver Transplantation Across AKI Groups, Per Estimated GFR (1) eGFR<30 ml/min/1.73 m² pre-LT and eGFR<30 ml/min/1.73 m² 4-12 weeks after LT (irreversible AKI=iAKI), (2) eGFR<30 ml/min/1.73 m² pre-LT and >50 ml/min/1.73 m² 4-12 weeks after LT (reversible AKI=rAKI), (3) eGFR>50 ml/min/1.73 m² prior to LT and >50 ml/min/1.73 m² 4-12 weeks after LT (normal=nAKI). (4) eGFR<30 ml/min/1.73 m² pre-LT and eGFR 30-50 ml/min/1.73 m² after LT (partial=pAKI).

FIG. 2B: Change In TIMP1 Levels Before and After Liver Transplantation Across AKI Groups, Per Estimated GFR (1) eGFR<30 ml/min/1.73 m² pre-LT and eGFR<30 ml/min/1.73 m² 4-12 weeks after LT (irreversible AKI=iAKI), (2) eGFR<30 ml/min/1.73 m² pre-LT and >50 ml/min/1.73 m² 4-12 weeks after LT (reversible AKI=rAKI), (3) eGFR>50 ml/min/1.73 m² prior to LT and >50 ml/min/1.73 m² 4-12 weeks after LT (normal=nAKI). (4) eGFR<30 ml/min/1.73 m² pre-LT and eGFR 30-50 ml/min/1.73 m² after LT (partial=pAKI).

FIG. 3: Receiver Operating Curve of the REVERSL Model (Age<57 Years, Absence Of Diabetes, OPN, TIMP) in Prediction of Renal Recovery After Liver Transplantation. AUC=0.78, 95% CI 0.63-0.93.

FIG. 4: Schematic illustrating algorithm for patient selection.

FIG. 5A: Absolute Osteopontin levels Across AKI Groups, Per Measured GFR (iothalamate clearance).

FIG. 5B: Absolute TIMP1 levels Across AKI Groups, Per Measured GFR (iothalamate clearance).

FIG. 6A: Change in Osteopontin levels before and after liver transplantation across AKI groups, per measured GFR (iothalamate clearance).

FIG. 6B: Change in TIMP1 levels before and after liver transplantation across AKI groups, per measured GFR (iothalamate clearance).

### DETAILED DESCRIPTION

Disclosed are methods for predicting, inhibiting, and treating chronic kidney disease (CKD) in a subject that that is preparing to undergo a liver transplant. The methods are described herein using several definitions, as set forth below and throughout the application.

As used in this specification and the claims, the singular forms “a,” “an,” and “the” include plural forms unless the context clearly dictates otherwise. For example, “a biomarker” should be interpreted to mean “one or more biomarkers,” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The presently disclosed methods relate to detecting biomarkers in a biological sample from a subject (e.g., a serum sample) that may be utilized to diagnose and/or prognose the subject, and optionally treat the diagnosed and/or prognosed subject based on the biomarker having been detected in the biological sample from the subject.

As used herein, the term “subject,” which may be used interchangeably with the terms “patient” or “individual,” refers to one who receives medical care, attention or treatment and may encompass a human subject. As used herein, the term “subject” is meant to encompass a person who has a liver disease or disorder and is in need of a liver transplant. The subject optionally may have or may be at risk for developing acute kidney injury, which may include reversible acute kidney injury (rAKI), partial acute kidney injury (pAKI), and irreversible acute kidney injury (iAKI), or the subject may not have acute kidney injury (nAKI).

The subject disclosed herein by have or may be at risk for developing chronic kidney disease (CKD), optionally where the chronic kidney disease occurs or develops after the subject has been administered a liver transplant. Kidney function may be assessed by techniques in the art including determining actual or estimated glomerular filtration rate either directly or via measuring protein biomarkers that are indicative of GFR such creatinine. CKD may be staged by GFR as follows: Stage 1 with normal or high GFR (GFR>90 mL/min); Stage 2 Mild CKD (GFR 60-89 mL/min); Stage 3A Moderate CKD (GFR 45-59 mL/min); Stage 3B Moderate CKD (GFR=30-44 mL/min); Stage 4 Severe CKD (GFR=15-29 mL/min); and Stage 5 End Stage CKD (GFR<15 mL/min).

The disclosed methods may include detecting a protein biomarker in a serum sample from a subject prior to the subject being administered a liver transplant procedure and/or after the subject has been administered the liver transplant procedure. Suitable protein markers for the disclosed methods may include but are not limited to one or more of the following biomarkers: osteopontin (OPN), inhibitor of metalloproteases 1 (TIMP1), alpha-1-microglobuline (AlMicro), beta-2-microglobulin (B2M), Calbindin, clusterin (CLU), Cystatin C, kidney injury molecule 1 (KIM-1), neutrophil gelatinase-associated lipocalin (NGAL), Tamm-Horsfall glycoprotein (THP), trefoil factor 3 (TFF3), and vascular endothelial growth fact (VEGF).

In some embodiments of the disclosed methods, the detected biomarkers are OPN and/or TIMP1. The amino acid sequence of human OPN is deposited in GenBank under accession number AAA59974. The amino acid sequence of human TIMP1 is deposited in GenBank under accession number CAG46779.

In some embodiments of the disclosed methods, OPN is detected in a serum sample of a subject at a concentration of at least about (or of no more than about) 10, 15, 20, 25, 30, 35, 40, 45, 50, 55, 60, 65, 70, 75, 80, 85, 90, 95, 100, 105, or 110 ng/ml, or within a range bounded by any two of these values. In some embodiments of the disclosed methods, TIMP1 is detected in a serum sample of a subject at a concentration of at least about (or of no more than about) 150, 200, 250, 300, 350, 400, 450, 500, 550, 600, 650, 700, 750, 800, 850, 900, 950, 1000, 1050, 1100, 1150, 1200, 1250, or 1300 ng/ml, or within a range bounded by any two of these values.

In some embodiments of the disclosed methods, the subject is administered treatment and/or the subject is not administered treatment. For example, in some embodiments of the disclosed methods, based on the detected levels of one or more biomarkers in a biological sample from the subject, the subject is administered treatment and/or the subject is not administered treatment.

In some embodiments of the disclosed methods, based on the detected levels of one or more biomarkers in a biological sample from the subject, the subject may be administered simultaneously a liver transplant and a kidney transplant. For example, in some embodiments of the disclosed methods, when the subject has a serum OPN concentration of less than about 110, 105, 100, 95, 90, 85, 80, 75, 70, 65, or 60 ng/ml, the subject may be administered simultaneously a liver transplant and a kidney transplant. In another example, in some embodiments of the disclosed methods, when the subject has a serum TIMP1 concentration of less than about 1300, 1250, 1200, 1150, 1100, 1050, 1000, 950, 900, 850, 800, 750, 700, 650, 600, 550, 500, 450, 400, or 350 ng/ml, the subject may be administered simultaneously a liver transplant and a kidney transplant.

In some embodiments of the disclosed methods, based on the detected levels of one or more biomarkers in a biological sample from the subject, the subject may be administered therapy to treat CKD and/or to inhibit the progression of CKD. For example, in some embodiments of the disclosed methods, when the subject has a serum OPN concentration of less than about 110, 105, 100, 95, 90, 85, 80, 75, 70, 65, or 60 ng/ml, the subject may be administered therapy to treat CKD and/or to inhibit the progression of CKD. In another example, in some embodiments of the disclosed methods, when the subject has a serum TIMP1 concentration of less than about 1300, 1250, 1200, 1150, 1100, 1050, 1000, 950, 900, 850, 800, 750, 700, 650, 600, 550, 500, 450, 400, or 350 ng/ml, the subject may be administered therapy to treat CKD and/or to inhibit the progression of CKD.

In some embodiments, of the disclosed methods, subjects that are preparing to undergo or that have undergone a liver transplant procedure may be administered immunosuppressive therapy, for example, to reduce the likelihood of tissue rejection. Immunosuppressive therapy may include administering immunosuppressive drugs to the subject such as, but not limited to, Prednisone, Cyclosporine, Tacrolimus, Mycophenolate Mofetil, Azathioprine or Rapamycin. However, some types of immunosuppressive drugs such as Cyclosporin and Tacrolimus have nephrotoxic effects depending upon their dose levels. In some embodiments of the disclosed methods, based on the detected levels of one or more biomarkers in a biological sample from the subject, the subject not be administered immunosuppressive drugs that are nephrotoxic.

In the disclosed methods, the protein biomarkers may be detected by methods known in the art. Methods for detecting protein biomarkers may include, but are not limited to, immunoassays.

The disclosed methods may be performed in order to diagnose or prognose renal function in a subject. As used herein the terms “diagnose” or “diagnosis” or “diagnosing” refer to distinguishing or identifying a disease, syndrome or condition or distinguishing or identifying a person having or at risk for developing a particular disease, syndrome or condition. As used herein the terms “prognose” or “prognosis” or “prognosing” refer to predicting an outcome of a disease, syndrome or condition. For example, the disclosed methods may be performed in order to prognose whether a subject's renal function is likely to improve after the subject receives a liver transplant.

The disclosed methods may include steps that include administering treatment. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration. In particular, the methods contemplated herein include treating a subject having or at risk for developing kidney disease.

The present methods may include detecting a biomarker in a biological sample from a subject. The term “sample” or “subject sample” is meant to include biological samples such as tissues and bodily fluids. “Bodily fluids” may include, but are not limited to, blood, serum, plasma, saliva, cerebral spinal fluid, pleural fluid, tears, lactal duct fluid, lymph, sputum, and semen. A sample may include nucleic acid, protein, or both.

As used herein, the term “detecting” may include qualitative detection (i.e., merely detecting the presence and/or absence of a biomarker in a biological sample) and/or quantitative detection (i.e., determining a concentration of a biomarker in a biological sample). As used herein, the term “assay” or “assaying” means qualitative or quantitative analysis or testing.

### ILLUSTRATIVE EMBODIMENTS

The following embodiments are illustrative and should not be interpreted to limit the scope of the claimed subject matter.

Embodiment 1. A method comprising: (a) detecting osteopontin (OPN) and/or tissue inhibitor of metalloproteinases 1 (TIMP1) in a serum sample from a subject; and subsequently (b) after the subject has been administered a liver transplant procedure, measuring renal function in the subject.

Embodiment 2. The method of embodiment 1, further comprising detecting OPN and tissue TIMP1 in a serum sample from the subject after the subject has been administered the liver transplant procedure.

Embodiment 3. The method of embodiment 1, wherein renal function is measured by determining or estimating glomerular filtration rate (GFR) in the subject.

Embodiment 4. The method of embodiment 1, further comprising measuring renal function in the subject prior to the subjecting being administering the liver transplant procedure.

Embodiment 5. The method of embodiment 1, further comprising quantifying amounts of OPN and/or TIMP1 in the serum sample.

Embodiment 6. The method of any of the foregoing embodiments, further comprising administering to the subject therapy to inhibit chronic kidney disease (CKD) or reduce the rate of progression of CKD.

Embodiment 7. The method of any of the foregoing embodiments, further comprising detecting creatinine in the serum sample prior to the liver transplant procedure and/or after the liver transplant procedure.

Embodiment 8. The method of any of the foregoing embodiments, further comprising administering therapy to the subject that reduces nephrotoxic effects of immunosuppressive therapy that is administered before, during, or after the liver transplant procedure.

Embodiment 9. A method comprising: (a) (i) detecting osteopontin (OPN) and/or tissue inhibitor of metalloproteinases 1 (TIMP1) in a serum sample from a subject, and (ii) measuring renal function in the subject; and subsequently (b) after the subject has been administered a liver transplant procedure, (i) detecting osteopontin (OPN) and/or tissue inhibitor of metalloproteinases 1 (TIMP1) in a serum sample from a subject, and (ii) measuring renal function in the subject.

Embodiment 10. The method of embodiment 9, wherein renal function is measured by determining or estimating glomerular filtration rate (GFR) in the subject.

Embodiment 11. The method of embodiment 9, further comprising quantifying amounts of OPN and/or TIMP1 in the serum sample.

Embodiment 12. The method of any of embodiments 9-11, further comprising administering to the subject therapy to inhibit chronic kidney disease (CKD) or reduce the rate of progression of CKD.

Embodiment 13. The method of any of embodiments 9-12, further comprising detecting creatinine in the serum sample prior to the liver transplant procedure and/or after the liver transplant procedure.

Embodiment 14. The method of any of embodiments 9-13, further comprising administering therapy to the subject that reduces nephrotoxic effects of immunosuppressive therapy that is administered before, during, or after the liver transplant procedure.

Embodiment 15. A kit comprising: (a) components for detecting osteopontin (OPN) and/or tissue inhibitor of metalloproteinases 1 (TIMP1) in a serum sample from a subject; and (b) components for measuring renal function in the subject.

### EXAMPLES

The following examples are illustrative and should not be interpreted to limit the scope of the claimed subject matter.

### Example 1

Reference is made to the Abstract submitted to the The Liver Meeting of the American Association for the Study of Liver Disease (AASLD) 2018, entitled “Discovery and Validation of an Early Post-Transplant Biomarker Model Predictive of Chronic Kidney Disease in Liver Transplant Recipients,” Levitsky, et al.

Background: A significant proportion of patients develop chronic kidney disease after liver transplantation (LT). We have developed clinical/protein models to predict glomerular filtration rate (GFR) deterioration in recipients with normal GFR early after LT.

Methods: Using independent cohorts (CTOT-14: a seven center prospective NIAID study and BUMC: a single center Baylor cohort), we analyzed protocol serum/plasma samples from recipients with preserved GFR (>60 ml/min/1.73 m²) at month 3 and had GFR deterioration vs. preservation at 1 year; year 5 GFR was also studied at BUMC. We also studied serial renal injury protein levels and GFR collected during the first year in the CTOT-14 cohort.

Year 1 GFR Model: Using the CTOT-14 cohort (n=61), a model was constructed from 7 clinical and 16 renal injury protein candidate variables. HCV infection and protein levels of β2-microglobulin and CD40 predicted early GFR deterioration, defined as >10% persistent decline over year 1 (AUC 0.814). We observed good validation of this model (AUC 0.801) in the BUMC sub-cohort (n=50) that met this same GFR definition of deterioration.

Year 5 GFR model: A model that predicts longer term, more advanced CKD (<45 ml/min/1.73 m²) was constructed based on month 3 BUMC samples (n=105). This model including age, male sex, cystatin-C and MCSF-1 (AUC 0.866) confirms that early renal injury protein levels can also predict late GFR deterioration.

Serial CTOT-14 Samples: Several proteins, including β2-microglobulin, al-microglobulin, cystatin C, trefoil factor 3, thrombomodulin, and uromodulin, correlated with deterioration in GFR over the first year post-LT in a linear mixed model.

Conclusions: We have developed and validated a clinical/protein model at 3 months post-LT (in preserved GFR recipients) that can predict renal deterioration at 1 year with reasonable accuracy. In addition, we report on a 5 year model and serial increases in several kidney injury proteins with GFR deterioration in the first year. These approaches may identify appropriate candidates for proactive renal sparing strategies early post LT.

### Example 2

Reference is made to the manuscript, Levitsky, et al., “External Validation of a Pretransplant Biomarker Model (REVERSE) Predictive of Renal Recovery After Liver Transplantation,” Hepatology. 2019 October; (70(4):13459-1359, 2019 May 28, the content of which is incorporated herein by reference in its entirety.

Title: External Validation of a Pre-Transplant Biomarker Model (REVERSL) Predictive of Renal Recovery after Liver Transplantation

Abstract

### BACKGROUND

in patients with end-stage liver disease, the ability to predict recovery of renal function following liver transplantation (LT) remains elusive. However, several important clinical decisions depend on whether renal dysfunction is recoverable after LT. We used a cohort of patients undergoing LT to independently validate a published pre-LT model predictive of post-transplant renal recovery (Renal Recovery Assessment at Liver Transplant [REVERSE]: high osteopontin [OPN] and tissue inhibitor of metalloproteinases-1 [TIMP-1] levels, age<57, no diabetes). Serum samples pre-LT and 4-12 weeks post-LI (n=117) were analyzed for kidney injury proteins from three groups of recipients: (1) estimated glomerular filtration rate (eGFR)<30 mL/minute/1.73 m² prior to and after Ur (irreversible acute kidney injury [AKI]), (2) eGFR<30 mL/minute/1.73 m² prior to LT and >50 mL/minute/1.73 m² after LT (reversible AKI [rAKI]) (3) eGFR>50 mL/minute/1.73 m² prior to and after LT (no AKI). In patients with elevated pre-LT serum levels of OPN and TIMP-1, recovery of renal function correlated with decreases in the level of both proteins. At 4 weeks post-LT (n=77 subset), the largest decline in OPN and TEMP-1 was seen in the rAKI group. Validation of the REVERSE model in this independent data set had high area under the curve (0.78) in predicting full post-LT renal recovery (sensitivity 0,86, specificity 0.6, positive predictive value 0.81, negative predictive value 0.69). Our eGFR findings were confirmed using measured GFR. Conclusion: The REVERSE model, derived from an initial training set combining plasma biomarkers and clinical characteristics, demonstrated excellent external validation performance characteristics in an independent patient cohort using serum samples. Among patients with kidney injury pre-LT, the predictive ability of this model may prove beneficial in clinical decision-making both prior to and following transplantation

### INTRODUCTION

Acute kidney injury (AKI) is common among patients with decompensated liver disease awaiting liver transplantation (LT) and is associated with a seven-fold increase in mortality (1). Pre-transplant renal dysfunction, particularly if not reversible after LT, is also an important predictor of chronic kidney disease (CKD), morbidity and mortality after LT (2-5). Accurate assessment of reversibility of pre-LT renal injury is important for several reasons. First, identification of patients with reversible renal injury may help adjudicate decisions for LT alone (LTA) as opposed to simultaneous liver-kidney transplantation (SLKT) (6, 7). This is important given the dramatic increase in SLKT, removing kidneys as usable organs for patients awaiting kidney transplant alone, as well as limitations of recently approved listing criteria that may not adequately identify acute versus chronic kidney disease (8, 9). On the other hand, among recipients undergoing LTA, identification of irreversible pre-transplant renal injury may allow for early initiation of more aggressive renal preservation strategies immediately after transplantation. This may mitigate future development or rate of progression of CKD after LT and its associated morbidity and mortality (5, 10).

Despite wide use, changes in serum creatinine or estimated glomerular filtration rates (eGFR) in isolation may not be sensitive to predict the degree or course of renal injury, particularly in patients with cirrhosis and malnutrition (11-14). Thus, there is significant interest in incorporation of other biomarkers that may enhance clinical prediction of renal recovery after liver transplantation (11, 15, 16). Osteopontin (OPN) is a multifaceted glycoprotein that is induced during inflammatory processes in AKI, contributes to renal tubular epithelial regeneration, and has been associated with recovery from critical care renal injury (17-19). Tissue inhibitor of metalloproteinases-1 (TIMP1) inhibits metalloproteinases which degrade extra cellular matrix in kidney injury regeneration and also appears to play a role in recovery of sepsis-related AKI (20, 21). We have previously developed a pre-LT predictive model, combining patient characteristics (age<57, no diabetes) and these two plasma proteins (high OPN, TIMP1) with a high area under the curve (AUC 0.82), capable of predicting post LT renal recovery (22). This model, now coined REVERSL (REnal recoVERy poSt Liver transplant), performed well independent of etiology of renal disease and other contributing factors.

In the current study, we sought to externally validate the ability of the REVERSL model to predict native renal recovery following LT using serum samples from an independent patient cohort, we hypothesized that this model will also predict renal recovery after LT in this cohort.

Methods

The primary objective of the study was to validate the model using post-LT RR, defined as recovery of renal function at 4-12 weeks post-LT. A secondary objective was to analyze changes in levels of several relevant proteins from the same array before and after LT compared to the degree of renal recovery, using both estimated and measured GFR.

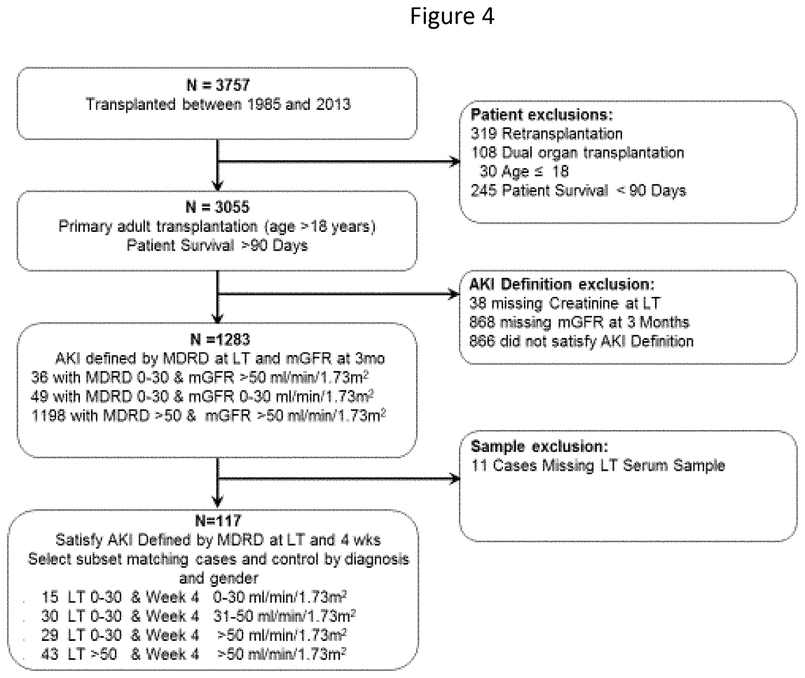

Patients and renal function: We examined all patients who underwent LTA between 1985 and 2013 at Baylor University Medical Center with stored serum samples obtained under an IRB-approved protocol before and 4-12 weeks after LTA (FIG. 4). For the primary objective, three discrete groups of patients were compared based on glomerular filtration as estimated by the Modification of Diet in Renal Disease Study equation (MDRD-4) from accepted KDIGO guidelines (23). These were identical to the designation of the groups from the discovery cohort (22): (1) eGFR<30 ml/min/1.73 m² pre-LT and eGFR<30 ml/min/1.73 m² 4-12 weeks after LT (irreversible AKI=iAKI), (2) eGFR<30 ml/min/1.73 m² pre-LT and >50 ml/min/1.73 m² 4-12 weeks after LT (reversible AKI=rAKI), (3) eGFR>50 ml/min/1.73 m² prior to LT and >50 ml/min/1.73 m² 4-12 weeks after LT (normal=nAKI). For the secondary objective, we included a fourth group who had partial reversal (pAKI): eGFR<30 ml/min/1.73 m² pre-LT and eGFR 30-50 ml/min/1.73 m² after LT. For estimated GFR estimates, the isotope dilution mass spectrometry reference measurement modified MDRD-4 equation was used. In sensitivity analysis, we replicated the analysis using discrete categories as assigned by protocol measured GFR.

The Baylor biorepository contains predesignated, protocolled serum sample collection for research. Of note, our prior findings were made on plasma samples as recommended by the manufacturer of the protein arrays. However the Baylor repository does not store plasma, and therefore we chose to carry out the study using serum samples instead. Further linkage is provided to a prospectively maintained database capturing inpatient, outpatient, and non-transplant related encounters in all listed patients before and after transplant, with seamless linkage to clinical data and long-term outcomes. Diagnoses were extracted in real time using administrative data, death data adjudicated by a single physician (GK) and manual chart review performed for data on relevant outcomes and discrepant or missing data. No donor organs were obtained from executed prisoners or other institutionalized persons.

Serum Protein Assays: Multi-analyte protein panels were performed on sera using a proprietary Luminex Bead technology and assay platform (14 protein Human KidneyMAP; Myriad RBM, Austin, Tex.). These proteins include cystatin C, kidney injury molecule (KIM)-1, NGAL, TFF3, VEGF, OPN, tissue inhibitor of metalloproteinase (TIMP), A1M, B2M, clusterin (CLU), glutathione S-transferase (GST)-a, Tamm Horsfall protein (THP), calbindin, and connective tissue growth factor (CTGF). In our prior study, we demonstrated that OPN and TIMP1, as compared to the other markers, were strongly associated with renal recovery and hence these were the focus of this validation study.

Statistical Analysis: First, we examined levels of several serum proteins selected based on our prior work. Specifically, we examined the relationship between OPN and TIMP1 with renal recovery. In a cross-sectional analysis, we first examined the association of baseline levels of the biomarkers prior to LT with subsequent renal recovery or lack thereof. Subsequently among patients with serial collections, we examined the absolute and percent change in biomarker level with subsets of renal recovery. Having established the association of OPN and TIMP1 with renal recovery, we independently validated the predictive model. Previously we have reported the results of model building with logistic regression analyses incorporating clinical (age, diabetes) and biomarker data (OPN, TIMP1). The modeling analysis yielded the optimal model shown below

$${log}_{e} ( \frac{p}{1 - p} ) = - 22.27 - 4.65 * AgeStatus - 2.28 * Diabetes + 1.17 * {log}_{e} OPN + 3.54 * {log}_{e} TIMP 1$$

where p is the occurrence probability of rAKI; age status=0 if age<57 years, or age status 1 if age>57 years; and diabetes: yes=1, no=0.

All statistical analyses utilized the SAS Enterprise Guide statistical package (version 9.4; SAS Institute Inc., Cary, N.C., USA) and R 3.3.2 Statistical Software (Foundation for Statistical Computing, Vienna, Austria). Statistical significance was set at p<0.05. The nominal P values were further adjusted for multiple comparisons with Bonferroni's correction. Nonparametric Tests: Wilcoxon Two-Sample Test (Kruskal-Wallis Test for >2 Groups) was used for comparison of continuous variables and Fishers Exact Test or Likelihood Ratio Chi-Square for dichotomous variables.

Results

Baseline characteristics: Between 1985 and 2013, there were 117 patients that met criteria. Table 1 provides details regarding the patients by assigned group of renal recovery.

TABLE 1
Basel line Pre-Transplant Characteristic by Category of Renal Recovery.

| | iAKI (n = 15) | pAKI (n = 30) | rAKI (n = 29) | nAKI (n = 43) | |
|---|---|---|---|---|---|
| eGFR | at LT: ≤30 | at LT: ≤30 | at LT: ≤30 | at LT: >50 | |
| (ml/min/1.73 m²) | Week 4-12: ≤30 | Week 4-12: 31-50 | Week 4-12: >50 | Week 4-12: >50 | p value |
| Age | 57 (48, 64) | 52 (46, 59) | 46 (31, 50) | 47 (40, 57) | 0.008 |
| MELD | 28 (23. 32) | 33 (26, 38) | 38 (33, 40) | 19 (17, 23) | <0.001 |
| Diabetes | 3 (20.0) | 4 (13.3) | 3 (10.3) | 3 (7.1) | 0.59 |
| Male | 8 (53.3) | 19 (63.3) | 16 (55.2) | 24 (55.8) | 0.89 |
| Caucasian | 13 (86.7) | 22 (73.3) | 20 (69.0) | 32 (74.4) | 0.92 |
| ETOH | 2 (13.3) | 4 (13.3) | 4 (13.8) | 7 (16.3) | 0.90 |
| HCV | 3 (20.0) | 8 (26.7) | 8 (27.6) | 12 (27.9) | |
| NASH/Cryptogenic | 4 (26.7) | 6 (20.0) | 5 (17.2) | 8 (18.6) | |
| eGFR at Listing | 26.8 (12.9, 129.5) | 33.2 (8.7, 104.5) | 28.7 (12.7, 120.9) | 93.7 (59.1, 151.3) | <.001 |
| eGFR at LT | 23.2 (10.3, 29.9) | 19.4 (8.7, 29.8) | 18.6 (10.6, 27.9) | 94.9 (57.3, 173.6) | <.001 |
| eGFR week 4 | 23.0 (11.4, 30.0) | 41.5 (31.1, 48.7) | 68.4 (54.2, 128.5) | 70.2 (53.6, 129.7) | <.001 |
| eGFR week 12 | 28.2 (6.1, 67.1) | 45.0 (19.6, 92.8) | 53.4 (21.7, 109.3) | 56.2 (20.4, 111.6) | 0.002 |
| Dialysis Listing to LT | 4 (26.7) | 7 (23.3) | 8 (27.6) | 0 (0.0) | <0.001 |
| Dialysis at LT | 2 (13.3) | 6 (20.0) | 7 (24.1) | 0 (0.0) | <0.001 |
| Dialysis Post LT | 8 (53.3) | 13 (43.3) | 13 (44.8) | 1 (2.3) | <.0001 |
| Total OR hours | 5.5 (3.8, 9.4) | 6.4 (4.5, 11.3) | 6.5 (4.3, 11.3) | 5.63 (4.2, 8.5) | 0.047 |
| # Blood transfusion | 4.0 (0.0, 18.0) | 6.5 (1.0, 28.0) | 7.5 (0.0, 21.0) | 4.0 (0.0, 11.0) | 0.020 |
| Rejection | 4 (26.7) | 5 (16.7) | 10 (34.5) | 15 (34.0) | 0.31 |
| Cyclosporine | 6 (40.0) | 16 (55.17) | 15 (51.72) | 11 (25.58) | 0.042 |
| Cyclosporine trough | 265.0 (172.0, 335.0) | 212.0 (180.0, 385.0) | 214.0 (165.0, 300.5) | 282.50 (236.0, 370.0) | 0.77 |
| Tacrolimus | 5 (33.3) | 13 (44.8) | 12 (41.4) | 31 (72.1) | 0.01 |
| Tacrolimus trough | 7.30 (5.8, 7.4) | 8.2 (5.6, 9.2) | 8.4 (5.1, 11.1) | 8.9 (6.2, 11.8) | 0.43 |
| Sirolimus | 5 (33.3) | 2 (6.9) | 3 (10.3) | 2 (4.7) | 0.045 |
| Sirolimus trough | 3.8 (3.4, 4.8) | 2.2 (2.2, 2.2) | 7.4 (4.6, 8.4) | 2.70 (2.7, 2.7) | 0.14 |

* Median and interquartile range (IQR) for continuous variables: Number and percentage (%) for categorical variables

The median MELD at LT was higher for patients with pAKI and rAKI as compared to patients with iAKI and nAKI. Patients with iAKI were older. Distribution by gender age, race and diagnosis was similar. Factors contributing to renal function pre-, peri-, and post-operatively had differences between nAKI and the AKI groups, but there was no clear difference between rAKI vs. iAKI, such as dialysis use, operating room time, blood transfusion requirement, and IS type and levels.

Baseline Pre-LT Biomarkers: Essentially all of the biomarkers in the panel were different between the groups and varied by the presence of pre-LT renal injury and the degree of renal recovery post-liver transplant (Table 2).

TABLE 2
Change in OPN and TIMP1 Levels by Category of Renal Recovery Before and After Transplantation.

| | iAKI (n = 15) | pAKI (n = 30) | rAKI (n = 29) | nAKI (n = 43) | |
|---|---|---|---|---|---|
| eGFR | at LT: ≤30 | at LT: ≤30 | at LT: ≤30 | at LT: >50 | |
| (ml/min/1.73 m²) | Week 4-12: ≤30 | Week 4-12: 31-50 | Week 4-12: >50 | Week 4-12: >50 | p value |
| A1Micro | 17.0 (6.40, 22.0) | 11.50 (6.60, 15.0) | 6.30 (4.20, 13.0) | 6.70 (4.10, 8.50) | <0.001 |
| B2M | 9.40 (6.90, 17.0) | 8.80 (6.60, 13.0) | 8.60 (6.70, 16.0) | 2.80 (2.30, 3.60) | <0.001 |
| Calbindin | 8.90 (8.90, 18.0) | 9.50 (8.90, 16.0) | 16.0 (9.10, 30.0) | 8.90 (8.90, 8.90) | <0.001 |
| CLU | 125.0 (105.0, 189.0) | 127.0 (95.0, 170.0) | 91.0 (63.0, 129.0) | 136.0 (104.0, 171.0) | 0.003 |
| Cystatin C | 2790.0 (2270.0, 3850.0) | 2850.0 (2270.0, 3220.0) | 2920.0 (2000.0, 4220.0) | 1160.0 (932.0, 1540.0) | <.01 |
| KIM-1 | 0.28 (0.23, 0.80) | 0.54 (0.35, 1.0) | 0.35 (0.24, 0.79) | 0.22 (0.10, 0.37) | <.01 |
| NGAL | 1140.0 (850.0, 1640.0) | 1375.0 (838.0, 2180.0) | 1970.0 (1190.0, 2770.0) | 435.0 (310.0, 695.0) | <.01 |
| OPN | 59.0 (39.0, 75.0) | 64.0 (52.0, 79.0) | 74.0 (57.0, 94.0) | 31.0 (25.0, 49.0) | <.01 |
| THP | 0.05 (0.03, 0.07) | 0.05 (0.04, 0.09) | 0.06 (0.05, 0.12) | 0.09 (0.06, 0.10) | 0.09 |
| TIMP1 | 361.0 (280.0, 570.0) | 552.50 (389.0, 880.0) | 719.0 (479.0, 1140.0) | 246.0 (195.0, 351.0) | <.01 |
| TTF3 | 0.67 (0.60, 3.0) | 0.95 (0.56, 1.60) | 1.20 (0.74, 2.50) | 0.18 (0.13, 0.23) | <.01 |
| VEGF | 248.0 (124.0, 337.0) | 231.0 (165.0, 407.0) | 236.0 (79.0, 388.0) | 153.0 (67.0, 261.0) | 0.054 |

Certain markers (OPN, TIMP1, Calbindin, cystatin C, NGAL, TFF-3) had the highest levels in the groups with rAKI as compared to iAKI group (p<0.01). Across all of the biomarkers, the lowest levels were in the nAKI group.

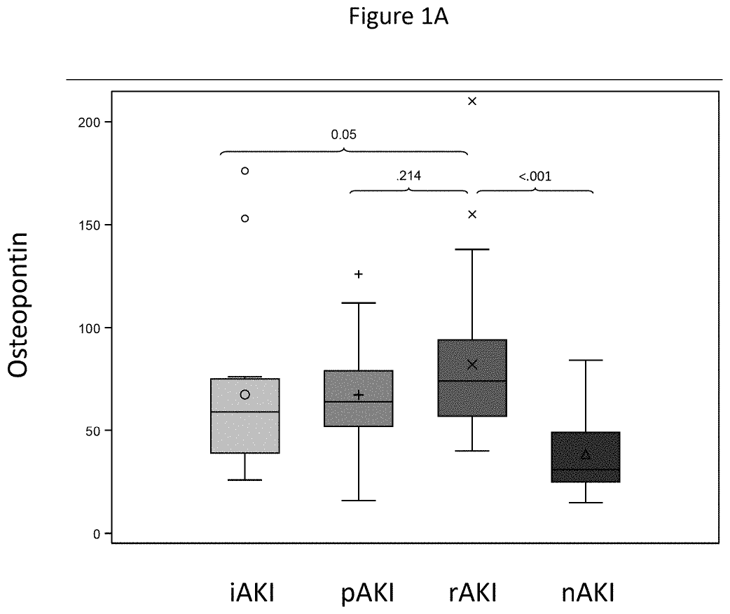

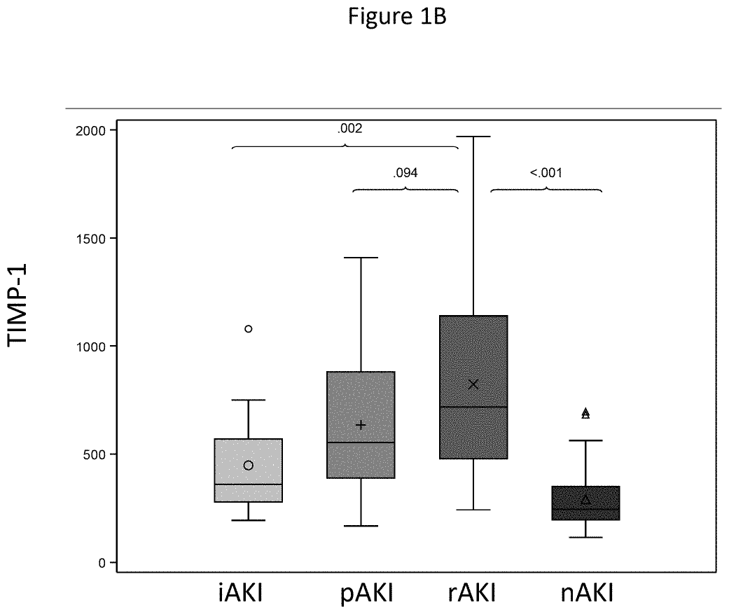

For the purpose of our validation study, patterns of elevation in OPN and TIMP1 are presented in FIGS. 1A and 1B. In pairwise comparisons, OPN was highest in rAKI 74.0 ng/mL (57.0, 94.0) as compared to nAKI (31.0 ng/mL (25.0, 49.0)) (p<0.01) and iAKI (59.0 ng/mL (39.00, 75.0)) (p=0.05). Similarly, TIMP1 was significantly higher in rAKI (719.0 ng/mL (479.0, 1140.00)) as compared to nAKI (246.00 ng/mL (195.0, 351.0) (<0.001) and iAKI (361.0 ng/mL (280.0, 570.0)) (p=0.002).

Serial Markers: Samples were available for testing before and after (4-12 weeks) LT in 77 patients (Table 3).

TABLE 3
Change in OPN and TIMP1 Levels by Category of Renal Recovery Before and After Transplantation.

| | iAKI (n = 6) | pAKI (n = 13) | rAKI (n = 15) | nAKI (n = 43) | |
|---|---|---|---|---|---|
| | at LT: ≤30 | at LT: ≤30 | at LT: ≤30 | at LT: >50 | |
| | Week 4-12: ≤30 | Week 4-12: 31-50 | Week 4-12: >50 | Week 4-12: >50 | p value |
| OPN | | | | | |
| At LT | 45.0 (26.0, 75.0) | 65.0 (26.0, 105.0) | 77.0 (40.0, 210.0) | 31.0 (19.0.76.0) | <.001 |
| Week 4-12 post-LT | 36.50 (23.0, 50.0) | 20.0 (5.70, 52.0) | 24.0 (4.70. 38.0) | 17.0 (5.80, 39.0) | 0.006 |
| Median difference | 16.0 (12.0, 39.0) | 44.0 (0.0, 87.0) | 62.0 (2.0, 173.0) | 16.0 (−9.0, 56.0) | <.001 |
| Median % difference | 26.25 (−46.2, 57.35) | 72.22 (0.0, 83.65) | 77.50 (5.0, 95.84) | 58.62 (−36.0, 84.15) | 0.010 |
| TIMP1 | | | | | |
| At LT | 381.0 (234.0, 751.0) | 582.0 (242.0, 1290.0) | 838.0 (301.0, 1650.0) | 246.0 (152.0, 562.0) | <.001 |
| Week 4-12 post-LT | 197.50 (152.0, 321.0) | 190.0 (131.0, 341.0) | 164.0 (119.0, 310.0) | 162.0 (114.0, 287.0) | 0.206 |
| Median difference | 186.0 (−37.0, 571.0) | 385.0 (38.0, 1055.0) | 599.0 (135.0, 1519.0) | 89.0 (−47.0, 383,0) | <.001 |
| Median % difference | 48.4 (−13.0, 76.03) | 70.60 (15.1, 82.65) | 73.04 (44.9, 92.06) | 37.15 (−34.6, 74.4) | <.001 |

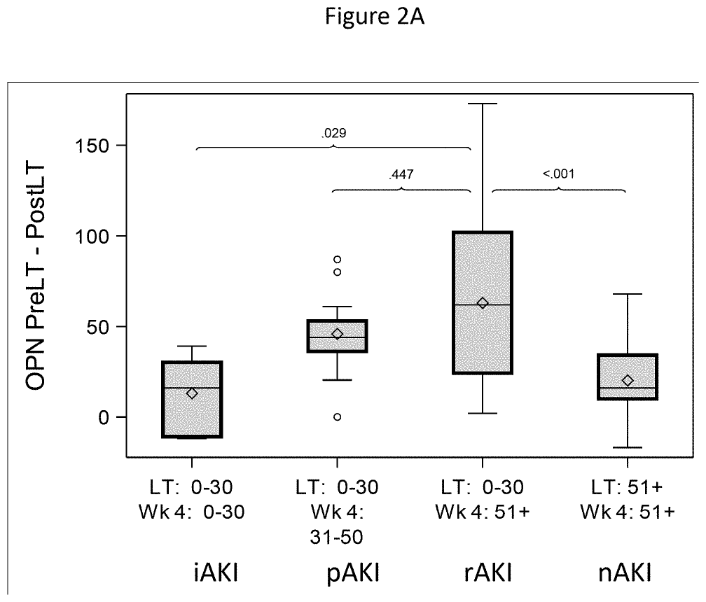

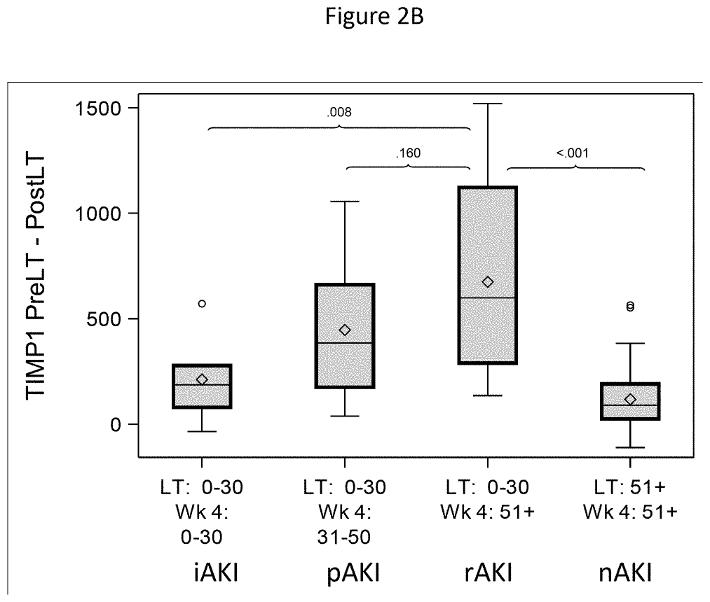

After LT, levels of OPN and TIMP1 were lower across all groups. However, the largest decline in levels for each marker was seen in the rAKI group followed by pAKI group for OPN and TIMP1, correlating with renal recovery (FIGS. 2A and 2B). The percent difference in OPN (26% vs 77.5%, p=0.01) and TIMP1 (48.4% vs. 73%, p=0.02) was lowest for iAKI as compared to rAKI.

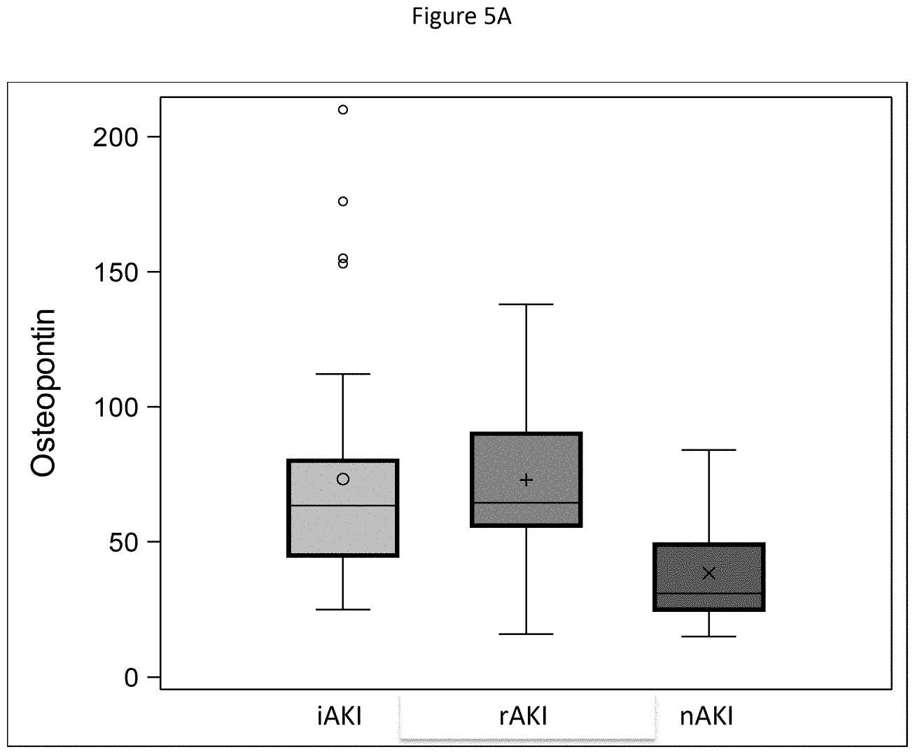

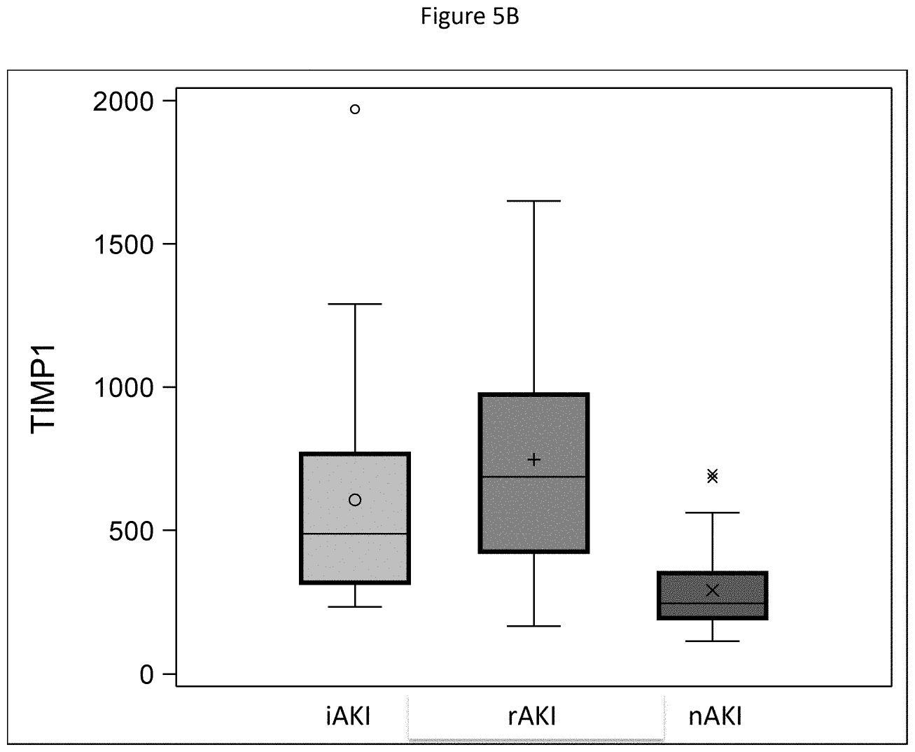

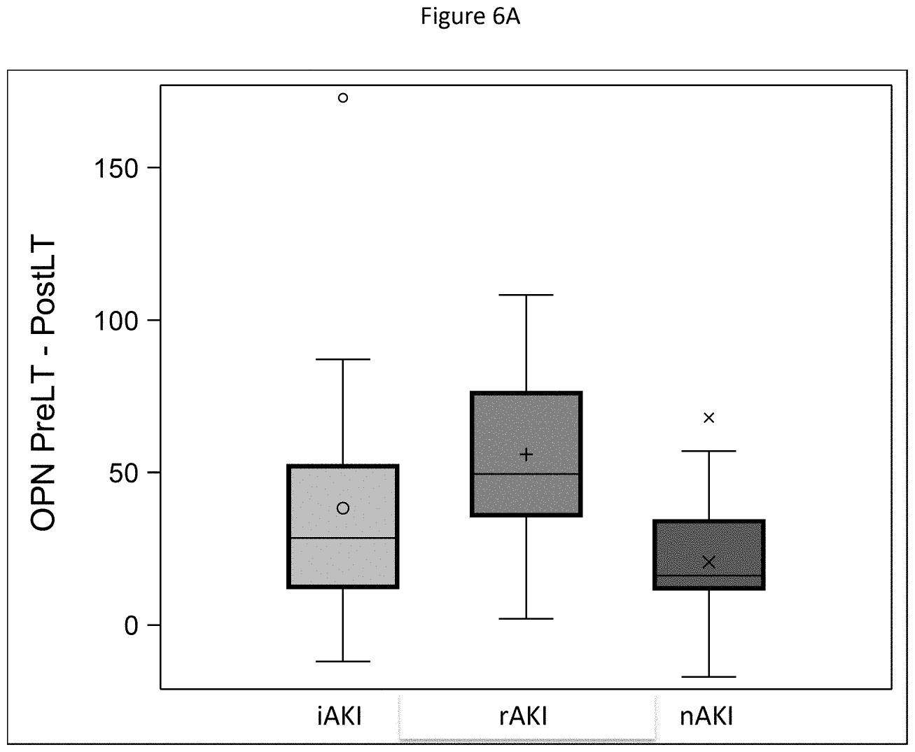

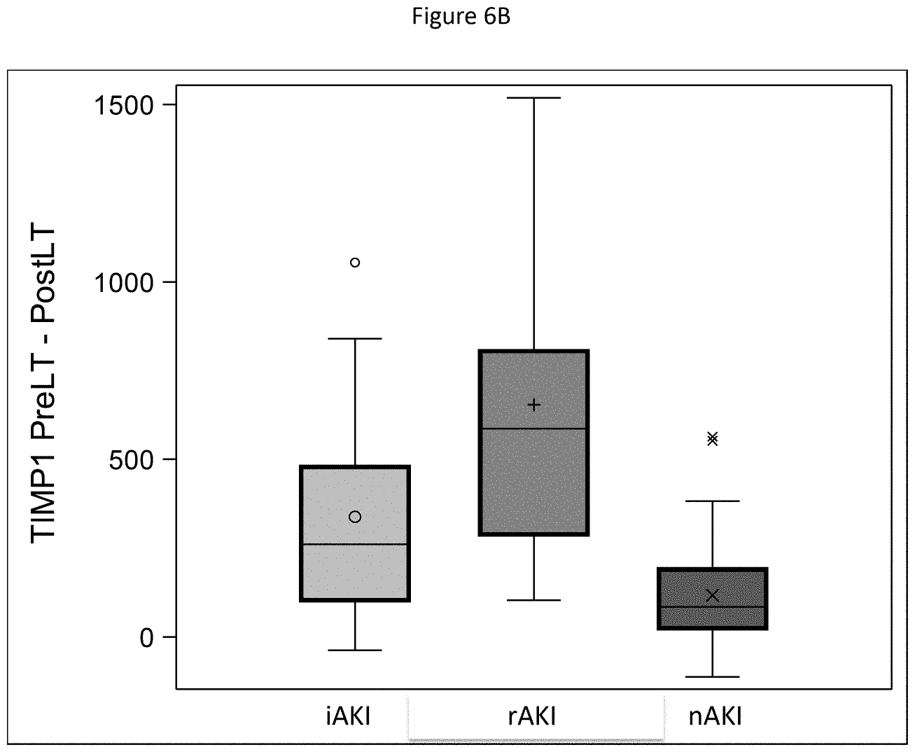

Measured GFR: Findings using mGFR correlated well with those using instead of eGFR (FIGS. 5A and 5B and FIGS. 6A and 6B).

REVERSL Model Validation: In our discovery cohort, using plasma, the model containing patient (age, diabetes) and biomarkers (OPN and TIMP1) performed as follows: the area under the curve (AUC) was 0.82. At the point of best prediction accuracy (0.81), the combined data model achieved a true positive rate of 0.78, a false positive rate of 0.15, a positive predictive value (PPV) of 0.88 and a negative predictive value (NPV) of 0.7 (5,22).

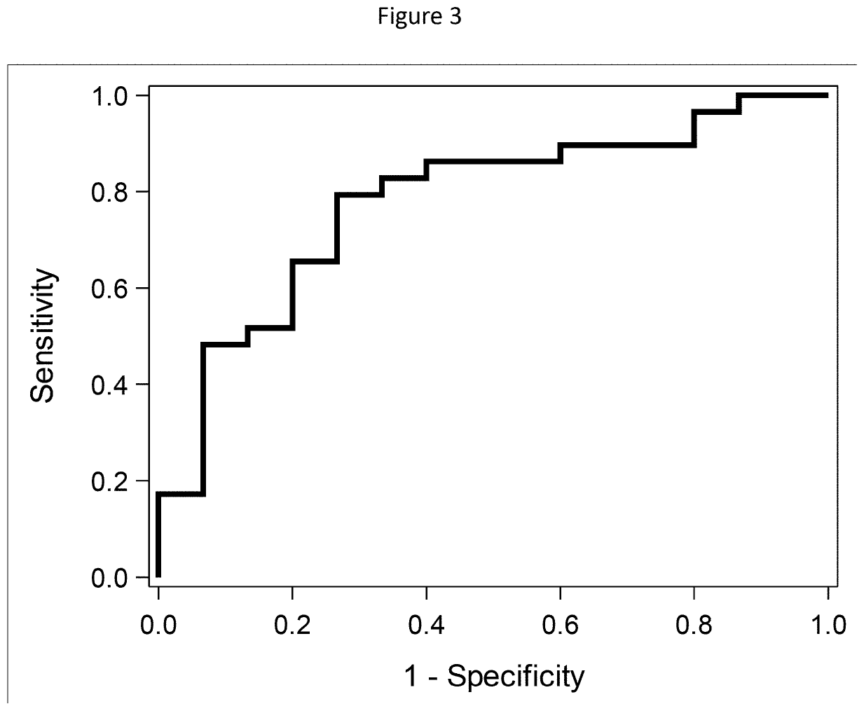

In our validation cohort, using serum instead of plasma (the Baylor biorepository did not have plasma stored, the AUC was (0.78) and had better fit than patient factors alone (c=0.66). We subsequently performed a logistic regression using the LR model cutoff variable (>0.8137) from the discovery model to predict renal recovery (22). Validation of the clinical/biomarker model in this independent external dataset had high AUC (c=0.78, 95% CI 0.63-0.93) in identifying full RR (rAKI) versus irreversible iAKI (sensitivity 0.86, specificity 0.6, PPV 0.81, and NPV 0.69 (FIG. 3).

### DISCUSSION

The ability to predict whether pre-transplant renal dysfunction will recover following LT has remained elusive and has largely been based on the chronicity of a low eGFR prior to LT including time on dialysis (24-26). However, the implications of non-recovery of renal function following LT are too important for ‘best guess’ speculation. Clinical decision-making in these complex cases include, but are not limited to: decision regarding simultaneous kidney transplant, choice of less effective but less nephrotoxic immunosuppressive agents, and informed choice by patients contemplating LT that relies on strong predictive information. For these reasons, we previously tested the ability of proteomic biomarkers in plasma (as recommended by the manufacturer of the protein arrays) to complement other clinical data to enhance accurate prediction in a single-center cohort (22). The performance metrics of a simple model (REVERSL) with serum biomarkers (osteopontin, TIMP1) were reasonably strong in this training set (AUC: 0.82). We now show that an external validation of this model in an independent patient cohort, and using serum instead of plasma, demonstrates similar performance, validating the clinical validity of our previous findings. Elevated pre-transplant OPN and TIMP1 declined rapidly after LT in patients with post-LT renal recovery.

Our approach is analogous to the evolution of modeling for other disease states. As an example, the risk of recurrence of hepatocellular carcinoma was initially based simply on size and number of tumors. However, further refinement of our understanding of the natural history has enabled incorporation of patient factors and biomarkers that improve prediction of survival and recurrence (27-29).

We believe that the model is now ready for clinical application to help predict the recovery of renal dysfunction in patients undergoing LT who may suffer from AKI for a number of reasons including hepato-renal syndrome (HRS), particularly type 2. Also, the model may become a useful tool in clinical trials that depend on the recovery on renal function. From a policy perspective, the model may need to be included in the decision to allow for a SLK rather than relying on the safety net (need reference). Moreover, decisions regarding less effective but less nephrotoxic immunosuppression agents long-term following LT may be facilitated by better understanding the natural history of the patient's renal function More specifically, the current UNOS system only allows for consideration of SLKT in patients with either prolonged AKI>6 weeks or ‘established’ CKD. However, these criteria are primarily arbitrary, leaving the decision-making to best guess estimates. For instance, prolonged AKI is defined as eGFR<30 ml/min/1.73 m² for >6 weeks, but this includes patients not on dialysis who may recover even after six weeks. Alternatively, the CKD definition only requires a single GFR measure of <60 ml/min/1.73 m² prior to 3 months and either regular dialysis or a recent single GFR<35 ml/min/1.73 m², the latter of which is relatively liberal in labeling patients with advanced CKD and candidates for SLKT. This is compounded by the fact that GFR measures are nearly universally creatinine-based and often overestimate true GFR. (11, 30).

In a previous report, we looked at recovery of native renal function in patients undergoing SLKT. Using a nucleotide renal scan, we were able to directly measure GFR in the transplanted kidney versus the native kidneys (7). We found that recovery of native renal function varied greatly among recipients of SLKT ranging from minimal recovery in the majority of patients, to greater and clinically meaningful recovery in the majority. In the current study, we have examined renal function that is strictly reflective of native renal recovery as none of these recipients underwent SLKT. Thus, we envision use of the model to identify recipients that are predicted to recover renal function post-transplant and, despite meeting criteria, may not need SLKT. Alternatively, the model could be used to inform changes in current policy if we can show that it is a better predictor for non-recovery than the current criteria. In addition, for patients not meeting SLKT criteria, centers could use the REVERSL model to select more long-term peri-/post-operative renal sparing immunosuppression strategies in patients predicted to not recover, so as to lessen the progression of CKD after LT. In contrast, in patients demonstrating a more likely recovery, the risk of using less effective immunosuppression could be lowered by reinstituting potentially nephrotoxic agents earlier post-transplant. Now that the REVERSL model has been validated, we believe that it should be used in clinical trials where recovery of renal function is relevant. In addition, the role of serial measurements of biomarkers in different trajectories of renal disease needs to be further studied in longer term post-LI management after this earlier period. We are currently analyzing similar biomarkers prospectively starting at 1 month post-LT in the NIAID CTOT-14 serial prospective study (31).

From a mechanistic perspective, our findings may also add new insights into the recovery of renal function in this and other patient populations. Clearly, there is a spectrum of functional and structural changes along the AKI/CKD continuum. Further some biomarkers may present evidence of perfusion, structural or regeneration-related changes (5, 6) (32)). OPN and TIMP1 markers may be surrogates of hemodynamic systemic and renal alterations that occur in decompensated liver disease. This is important given that many of the causes of AKI pre-LT may be potentially reversible (33). More specifically, OPN and TIMP1 appear to have a role in prediction of reversible renal injury in several disease states (34-37). As a pro-inflammatory cytokine, OPN helps modulate the inflammatory response and is expressed in a broad range of tissues (17). With kidney damage or inflammation, expression on OPN is upregulated (38). In critically ill patients with AM, OPN levels were high and correlated with renal recovery in serial measurements, remarkably similar to our two cohorts (18). In addition, OPN may be an earlier marker of AKI as compared to other markers such as cystatin C (39). OPN expression is upregulated in all nephrons after acute injury and is associated with regeneration of renal tubular epithelia after acute tubular necrosis (38, 40). In mouse models of HRS, induction of hepatic injury was paralleled by tubular injury, impaired microvascular flow, and increased levels of OPN (41). The role of OPN may be not be entirely clear as it is also associated with increased mortality in patients after discontinuation of RRT (42), although a large change in OPN levels was associated with a higher chance of being independent from RRT (18). This parallels the rapid reduction in this biomarker with renal recovery after LT in our cohort.

Similarly, TIMP1 may play several roles in AKI related to decompensated cirrhosis and renal repair. On the one hand, TIMP1 may modulate matrix metalloproteinases. Kidney injury regeneration involves remodeling of the extracellular matrix and matrix metalloproteinases contribute to tubulogenesis and recovery; elevated levels of TIMP1 may be associated with both acute and chronic changes (43). We had previously postulated that TIMP1 levels increase in response to elevation in matrix metalloproteinases in renal recovery mechanisms, and now this is supported by the fact that TAW′ levels decline with recovery and stay elevated with irreversible injury, like OPN. On the other hand, elevated levels of TIMP1 were observed in sepsis-associated acute kidney injury (SA-AKI) (20). TIMP1 may also be reflective of severity of liver disease and hemodynamic related changes in cirrhosis (44).

Our study has several strengths. Performance characteristics of the reported model were robust in independent validation, which typically leads to much lower accuracy than discovery. Similar results were obtained despite use of variety of immunosuppressive agents and clinical care protocols between the discovery and validation cohorts. Moreover, in the original study, we used plasma samples as recommended by the manufacturer. However, plasma is typically less available than serum and not surprisingly, the Baylor biorepository did not contain stored plasma. We therefore used sera instead. This is important given the validation of the assay in a different blood compartment. We were able to utilize the strength of protocol-collected samples before and after LT with tight linkage to clinical data in both cohorts. Another strength of this validation study is that we confirmed the correlation between the biomarker levels and true measured GFR before and after LT. This is important because while serum creatinine-based GFR estimating equations are used as standard of care, they may be imprecise at low GFR. Given that measured GFR is not readily available or practically used in serial fashion, we utilized MDRD-4 to align with the definition and because it was used in the discovery cohort, reflecting real world risk stratification. However, even when limited to mGFR in the validation cohort, the results were similar.

Our study also has several limitations. We recognize that performance of biomarkers may depend on degree of liver dysfunction or portal hypertension, as some of them are produced or metabolized by the liver. In the discovery cohort, we did not find a correlation between degree of liver failure and biomarker levels, and the MELD range in both cohorts were similar (22). However, to similarly address this issue in the validation cohort, we performed additional analyses demonstrating that elevated OPN and TIMP1 levels correlated with increasing bilirubin (OPN r=0.31, p<0.01; TIMP1 r=0.4, p<0.01) but not international normalized ratios (OPN r=0.25, p-0.07; TIMP1 r=0.15, p=0.3). This suggests that advanced liver synthetic dysfunction may not uniformly account for elevated biomarker levels observed in the study. Another issue is that cirrhotic patients with AKI may lie on an AKI-CKD spectrum (11, 45), and the presence of CKD, clinical or subclinical, may modify biomarker profiles (46). We were unable to examine for more definitive signs of CKD as renal biopsies were not performed, nor were we able to clearly determine the cause or true chronicity of renal dysfunction (47, 48). Finally, several other factors may contribute to AM including insults such as bleeding, nephrotoxic medications, intraoperative and post-operative aspects that cannot be fully accounted for in pre-LT predictive models. To address this, we did not find differences in such factors available in the data set, such as dialysis use, operating room time, blood transfusions, and immunosuppression, between those with reversible and irreversible injury. In addition, incorporation of kidney disease risk factors (e.g. age and DM) as well as having a marker panel agnostic to etiology of AKI is appealing.

In summary, we believe that the demonstration of sustained predictive performance of the REVERSL model in an external validation using an independent patient cohort should lead to a number of clinical applications that leverage an improved ability to predict recovery of native renal function in LT recipients with compromised renal function at the time of transplant. We also believe that mechanistic insights derived from this study will inform further studies focused on the causes of renal dysfunction in this and other patient populations.

### REFERENCES

1. Fede G, D'Amico G, Arvaniti V, Tsochatzis E, Germani G, Georgiadis D, Morabito A, et al. Renal failure and cirrhosis: A systematic review of mortality and prognosis. Journal of hepatology 2011.
2. Sanchez E Q, Gonwa T A, Levy M F, Goldstein R M, Mai M L, Hays S R, Melton L B, et al. Preoperative and perioperative predictors of the need for renal replacement therapy after orthotopic liver transplantation. Transplantation 2004; 78:1048-1054.
3. Gonwa T A, McBride M A, Anderson K, Mai M L, Wadei H, Ahsan N. Continued influence of preoperative renal function on outcome of orthotopic liver transplant (OLTX) in the US: where will MELD lead us? Am J Transplant 2006; 6:2651-2659.
4. Sharma P, Schaubel D E, Guidinger M K, Merion R M. Effect of pretransplant serum creatinine on the survival benefit of liver transplantation. Liver Transpl 2009; 15:1808-1813.
5. Levitsky J, O'Leary J G, Asrani S, Sharma P, Fung J, Wiseman A, Niemann C U. Protecting the Kidney in Liver Transplant Recipients: Practice-Based Recommendations From the American Society of Transplantation Liver and Intestine Community of Practice. Am J Transplant 2016; 16:2532-2544.
6. O'Leary J G, Levitsky J, Wong F, Nadim M K, Charlton M, Kim W R. Protecting the Kidney in Liver Transplant Candidates: Practice-Based Recommendations From the American Society of Transplantation Liver and Intestine Community of Practice. Am J Transplant 2016; 16:2516-2531.
7. Levitsky J, Baker T, Ahya S N, Levin M L, Friedewald J, Gallon L, Ho B, et al. Outcomes and native renal recovery following simultaneous liver-kidney transplantation. Am J Transplant 2012; 12:2949-2957.
8. Kim W R, Lake J R, Smith J M, Schladt D P, Skeans M A, Harper A M, Wainright J L, et al. OPTN/SRTR 2016 Annual Data Report: Liver. Am J Transplant 2018; 18 Suppl 1:172-253.
9. Chan H L, Chim A M, Lau J T, Hui A Y, Wong V W, Sung J J. Evaluation of model for end-stage liver disease for prediction of mortality in decompensated chronic hepatitis B. Am J Gastroenterol 2006; 101:1516-1523.
10. Allen A M, Kim W R, Therneau T_{M}, Larson J J, Heimbach J K, Rule A D.

Chronic kidney disease and associated mortality after liver transplantation—A time-dependent analysis using measured glomerular filtration rate. J Hepatol 2014; 61:286-292.
11. Durand F, Francoz C, Asrani S K, Khemichian S, Pham T A, Sung R S, Genyk Y S, et al. Acute Kidney Injury after Liver Transplantation. Transplantation 2018.
12. Carrier P, Debette-Gratien M, Essig M, Loustaud-Ratti V. Beyond serum creatinine: which tools to evaluate renal function in cirrhotic patients? Hepatol Res 2018.
13. Francoz C, Nadim M K, Baron A, Prie D, Antoine C, Belghiti J, Valla D, et al. Glomerular filtration rate equations for liver-kidney transplantation in patients with cirrhosis: validation of current recommendations. Hepatology 2014; 59:1514-1521.
14. Nadim M K, Kellum J A, Davenport A, Wong F, Davis C, Pannu N, Tolwani A, et al. Hepatorenal syndrome: the 8th International Consensus Conference of the Acute Dialysis Quality Initiative (ADQI) Group. Crit Care 2012; 16:R23.
15. Jaques D A, Spahr L, Berra G, Poffet V, Lescuyer P, Gerstel E, Garin N, et al. Biomarkers for acute kidney injury in decompensated cirrhosis: A Prospective Study. Nephrology (Carlton) 2018.
16. Allen A M, Kim W R, Larson J J, Colby C, Therneau T_{M}, Rule A D. Serum Cystatin C as an Indicator of Renal Function and Mortality in Liver Transplant Recipients. Transplantation 2015; 99:1431-1435.
17. Icer M A, Gezmen-Karadag M. The multiple functions and mechanisms of osteopontin. Clin Biochem 2018.
18. Lorenzen J M, Hafer C, Faulhaber-Walter R, Kumpers P, Kielstein J T, Haller H, Fliser D. Osteopontin predicts survival in critically ill patients with acute kidney injury. Nephrol Dial Transplant 2011; 26:531-537.
19. Basile D P, Fredrich K, Alausa M, Vio C P, Liang M, Rieder M R, Greene A S, et al. Identification of persistently altered gene expression in the kidney after functional recovery from ischemic acute renal failure. Am J Physiol Renal Physiol 2005; 288:F953-963.
20. Bojic S, Kotur-Stevuljevic J, Kalezic N, Stevanovic P, Jelic-Ivanovic Z, Bilanovic D, Memon L, et al. Diagnostic Value of Matrix Metalloproteinase-9 and Tissue Inhibitor of Matrix Metalloproteinase-1 in Sepsis-Associated Acute Kidney Injury. Tohoku J Exp Med 2015; 237:103-109.
21. Kusaka M, Kuroyanagi Y, Ichino M, Sasaki H, Maruyama T, Hayakawa K, Shiroki R, et al. Serum tissue inhibitor of metalloproteinases 1 (TIMP-1) predicts organ recovery from delayed graft function after kidney transplantation from donors after cardiac death. Cell Transplant 2010; 19:723-729.
22. Levitsky J, Baker T B, Jie C, Ahya S, Levin M, Friedewald J, Al-Saden P, et al. Plasma protein biomarkers enhance the clinical prediction of kidney injury recovery in patients undergoing liver transplantation. Hepatology 2014; 60:2017-2026.
23. Stevens P E, Levin A, Kidney Disease: Improving Global Outcomes Chronic Kidney Disease Guideline Development Work Group M. Evaluation and management of chronic kidney disease: synopsis of the kidney disease: improving global outcomes 2012 clinical practice guideline. Ann Intern Med 2013; 158:825-830.
24. Nadim M K, Sung R S, Davis C L, Andreoni K A, Biggins S W, Danovitch G M, Feng S, et al. Simultaneous liver-kidney transplantation summit: current state and future directions. Am J Transplant 2012; 12:2901-2908.
25. Eason J D, Gonwa T A, Davis C L, Sung R S, Gerber D, Bloom R D. Proceedings of Consensus Conference on Simultaneous Liver Kidney Transplantation (SLK). Am J Transplant 2008; 8:2243-2251.
26. Charlton M R, Wall W J, Ojo A O, Gines P, Textor S, Shihab F S, Marotta P, et al. Report of the first international liver transplantation society expert panel consensus conference on renal insufficiency in liver transplantation. Liver transplantation: official publication of the American Association for the Study of Liver Diseases and the International Liver Transplantation Society 2009; 15:S1-34.
27. Yang J D, Kim W R, Park K W, Chaiteerakij R, Kim B, Sanderson S O, Larson J J, et al. Model to estimate survival in ambulatory patients with hepatocellular carcinoma. Hepatology 2012; 56:614-621.
28. Giard J M, Mehta N, Dodge J L, Roberts J P, Yao F Y. Alpha-Fetoprotein Slope>7.5 ng/mL per Month Predicts Microvascular Invasion and Tumor Recurrence After Liver Transplantation for Hepatocellular Carcinoma. Transplantation 2018; 102:816-822.
29. Mehta N, Heimbach J, Harnois D M, Sapisochin G, Dodge J L, Lee D, Burns J M, et al. Validation of a Risk Estimation of Tumor Recurrence After Transplant (RETREAT) Score for Hepatocellular Carcinoma Recurrence After Liver Transplant. JAMA Oncol 2017; 3:493-500.
30. Asrani S K, Jennings L, Nadim M K, Levitsky J, Trotter J F, Klintmalm G. A novel equation, GRAIL (GfR Assessment In Liver disease) on the waitlist predicts need for kidney after liver transplantation. Hepatology 2017; 66:16A-16A.
31. CTOT-14: Discovery and Validation of Proteogenomic Biomarker Panels in a Prospective Serial Blood and Urine Monitoring Study of Liver Transplant Recipients—Transplant Proteogenomics. ClinicalTrials.gov NCT01672164.
32. Maiwall R, Sarin S K, Moreau R. Acute kidney injury in acute on chronic liver failure. Hepatol Int 2016; 10:245-257.
33. Martin-Llahi M, Guevara M, Torre A, Fagundes C, Restuccia T, Gilabert R, Sola E, et al. Prognostic importance of the cause of renal failure in patients with cirrhosis. Gastroenterology 2011; 140:488-496 e484.
34. Taub P R, Borden K C, Fard A, Maisel A. Role of biomarkers in the diagnosis and prognosis of acute kidney injury in patients with cardiorenal syndrome. Expert Rev Cardiovasc Ther 2012; 10:657-667.
35. Phillips J A, Holder D J, Ennulat D, Gautier J C, Sauer J M, Yang Y, McDuffie E, et al. Rat Urinary Osteopontin and Neutrophil Gelatinase-Associated Lipocalin Improve Certainty of Detecting Drug-Induced Kidney Injury. Toxicol Sci 2016; 151:214-223.
36. Kashiwagi E, Tonomura Y, Kondo C, Masuno K, Fujisawa K, Tsuchiya N, Matsushima S, et al. Involvement of neutrophil gelatinase-associated lipocalin and osteopontin in renal tubular regeneration and interstitial fibrosis after cisplatin-induced renal failure. Exp Toxicol Pathol 2014; 66:301-311.
37. Won A J, Kim S, Kim Y G, Kim K B, Choi W S, Kacew S, Kim K S, et al. Discovery of urinary metabolomic biomarkers for early detection of acute kidney injury. Mol Biosyst 2016; 12:133-144.
38. Xie Y, Sakatsume M, Nishi S, Narita I, Arakawa M, Gejyo F. Expression, roles, receptors, and regulation of osteopontin in the kidney. Kidney Int 2001; 60:1645-1657.
39. Wunnapuk K, Liu X, Gobe G C, Endre Z H, Peake P W, Grice J E, Roberts M S, et al. Kidney biomarkers in MCPA-induced acute kidney injury in rats: reduced clearance enhances early biomarker performance. Toxicol Lett 2014; 225:467-478.
40. Xie Y, Nishi S, Iguchi S, Imai N, Sakatsume M, Saito A, Ikegame M, et al. Expression of osteopontin in gentamicin-induced acute tubular necrosis and its recovery process. Kidney Int 2001; 59:959-974.
41. Varga Z V, Erdelyi K, Paloczi J, Cinar R, Zsengeller Z K, Jourdan T, Matyas C, et al. Disruption of renal arginine metabolism promotes kidney injury in hepatorenal syndrome. Hepatology 2018.
42. Yang T, Sun S, Zhao Y, Liu Q, Han M, Lin L, Su B, et al. Biomarkers upon discontinuation of renal replacement therapy predict 60-day survival and renal recovery in critically ill patients with acute kidney injury. Hemodial Int 2018; 22:56-65.
43. Kwiatkowska E, Domanski L, Bober J, Safranow K, Romanowski M, Pawlik A, Kwiatkowski S, et al. Urinary Metalloproteinases-9 and -2 and Their Inhibitors TIMP-1 and TIMP-2 are Markers of Early and Long-Term Graft Function After Renal Transplantation. Kidney Blood Press Res 2016; 41:288-297.
44. Busk T_{M}, Bendtsen F, Nielsen H J, Jensen V, Brunner N, Moller S. TIMP-1 in patients with cirrhosis: relation to liver dysfunction, portal hypertension, and hemodynamic changes. Scand J Gastroenterol 2014; 49:1103-1110.
45. Chawla L S, Bellomo R, Bihorac A, Goldstein S L, Siew E D, Bagshaw S M, Bittleman D, et al. Acute kidney disease and renal recovery: consensus report of the Acute Disease Quality Initiative (ADQI) 16 Workgroup. Nat Rev Nephrol 2017; 13:241-257.
46. Succar L, Pianta T J, Davidson T, Pickering J W, Endre Z H. Subclinical chronic kidney disease modifies the diagnosis of experimental acute kidney injury. Kidney Int 2017; 92:680-692.
47. Asrani S K, Kim W R, Heimbach J H, Rosen C B, Berndt M M, Wiesner R H, Kim B, et al. Acute Tubular Necrosis in present in the majority of patients undergoing liver transplantation. Hepatology 2011; 54:159A.
48. Nadim M K, Genyk Y S, Tokin C, Fieber J, Ananthapanyasut W, Ye W, Selby R. Impact of etiology of acute kidney injury on outcomes following liver transplantation: Acute tubular necrosis versus hepatorenal syndrome. Liver Transpl 2012.

1. A method comprising:
(a) detecting osteopontin (OPN) and/or tissue inhibitor of metalloproteinases 1 (TIMP1) in a serum sample from a subject; and subsequently
(b) after the subject has been administered a liver transplant procedure, measuring renal function in the subject.

2. The method of claim 1, further comprising detecting OPN and tissue TIMP1 in a serum sample from the subject after the subject has been administered the liver transplant procedure.

3. The method of claim 1, wherein renal function is measured by determining or estimating glomerular filtration rate (GFR) in the subject.

4. The method of claim 1, further comprising measuring renal function in the subject prior to the subjecting being administering the liver transplant procedure.

5. The method of claim 1, further comprising quantifying amounts of OPN and/or TIMP1 in the serum sample.

6. The method of claim 1, further comprising administering to the subject therapy to inhibit chronic kidney disease (CKD) or reduce the rate of progression of CKD.

7. The method of claim 1, further comprising detecting creatinine in the serum sample prior to the liver transplant procedure and/or after the liver transplant procedure.

8. The method of claim 1, further comprising administering therapy to the subject that reduces nephrotoxic effects of immunosuppressive therapy that is administered before, during, or after the liver transplant procedure.

9. A method comprising:
(a) (i) detecting osteopontin (OPN) and/or tissue inhibitor of metalloproteinases 1 (TIMP1) in a serum sample from a subject, and (ii) measuring renal function in the subject; and subsequently
(b) after the subject has been administered a liver transplant procedure, (i) detecting osteopontin (OPN) and/or tissue inhibitor of metalloproteinases 1 (TIMP1) in a serum sample from a subject, and (ii) measuring renal function in the subject.

10. The method of claim 9, wherein renal function is measured by determining or estimating glomerular filtration rate (GFR) in the subject.

11. The method of claim 9, further comprising quantifying amounts of OPN and/or TIMP1 in the serum sample.

12. The method of claim 9, further comprising administering to the subject therapy to inhibit chronic kidney disease (CKD) or reduce the rate of progression of CKD.

13. The method of claim 9, further comprising detecting creatinine in the serum sample prior to the liver transplant procedure and/or after the liver transplant procedure.

14. The method of claim 9, further comprising administering therapy to the subject that reduces nephrotoxic effects of immunosuppressive therapy that is administered before, during, or after the liver transplant procedure.

15. A kit comprising:
(a) components for detecting osteopontin (OPN) and/or tissue inhibitor of metalloproteinases 1 (TIMP1) in a serum sample from a subject; and
(b) components for measuring renal function in the subject.
